(19) United States
(12) Patent Application Publication
(10) Pub. No.: US 2013/0066263 A1
(43) Pub. Date: Mar. 14, 2013

(54) MICROJET DRUG DELIVERY SYSTEM AND MICROJET INJECTOR

(71) Applicants: Jae-Ick Yoh, Seoul (KR); Tae-Hee Han, Seoul (KR)

(72) Inventors: Jae-Ick Yoh, Seoul (KR); Tae-Hee Han, Seoul (KR)

(73) Assignee: SNU R&DB FOUNDATION, Seoul (KR)

(21) Appl. No.: 13/634,936

(22) PCT Filed: Mar. 16, 2011
(86) PCT No.: PCT/KR2011/001834

§ 371 Date: Nov. 28, 2012

(30) Foreign Application Priority Data
Mar. 16, 2010 (KR) 10-2010-0023210
Jun. 15, 2010 (KR) 10-2010-0056637
Mar. 16, 2011 (KR) 10-2011-0023331

(51) Int. Cl. A61M 5/30

(52) U.S. Cl. CPC A61M 5/30
USPC 604/70

(57) ABSTRACT

The present invention relates to a microjet drug delivery system for microjet spraying a drug solution stored inside to inject the same into the bodily tissue of a person to be operated, and a microjet injector. The microjet injector comprises: a pressure chamber completely filled with the liquid for propelling pressure, having an accommodation space with one side opened; an elastic film, which is a film member made of an elastic material, arranged so as to form a closed space by closing the opened one side of the pressure chamber; a drug chamber for accommodating a drug solution in a predetermined inner space, provided adjacent to the pressure chamber with interposing the elastic film therebetween; and a microjet nozzle communicating with the inner space of the pressure chamber so as to be formed as a channel for allowing the drug solution stored inside the pressure chamber to be microjet sprayed to the outside. The microjet drug delivery system provided by the present invention comprises: the microjet injector; an energy focusing device for generating bubbles in the liquid for propelling pressure stored in the pressure chamber by applying a concentrated energy to the liquid for propelling pressure; and a connecting adaptor for selectively detachably coupling the microjet injector to the energy focusing device.

### TECHNICAL FIELD

The present invention relates to a drug delivery system for administering a drug into bodily tissues of a patient and a microjet injector for use in the same, and more particularly, to a novel type of a needle-free drug delivery system and an injector, in which a injection drug is sprayed at high speed in the form of a liquid microjet so as to penetrate into the skin of the patient instead of injecting the drug into the bodily tissues of the patient using a syringe needle or the like, so that the drug solution can rapidly and accurately penetrate into the bodily tissues of the patient while alleviating the pain felt during the injection.

### BACKGROUND ART

In general, a variety of drug delivery systems or methods have been applied as a method for parenterally administering a treatment drug into a patient's body in a medical field. In these drug delivery systems, the most commonly used method is an intracutaneous injection method employing a conventional syringe in which as well known, a syringe needle having a sharp tip pierces the skin tissue of a patient and a drug is pressurized so as to be injected into the patient's body through the needle.

The above intracutaneous injection method has an advantage in that since the drug is directly injected into the patient's body so that it can be very effectively administered in vivo, in that the injection is relatively simple and convenient, and the medical cost burden is greatly reduced. However, such an intracutaneous injection method entails a great shortcoming in that the patient suffers from an inconvenience of having to feel a pain during the injection. Besides, the intracutaneous injection method encounters a drawback in that a wound is caused by the use of a syringe needle, leading to a risk of wound infection. In addition, this intracutaneous injection method involves big problems in that since the re-use of the retractable needle-type syringe is difficult due to hygienic reason, it is thrown away as a disposable item, resulting in waste of resources, and in that an error may occur depending on proficiency of an operator performing the injection treatment.

Due to the drawbacks of the above-mentioned conventional intracutaneous injection method, many research have been made to develop a novel type of drug delivery system as a substitute for the conventional intracutaneous injection method. In an attempt to develop the novel type drug delivery system, there has been proposed a needle-less drug delivery system which injects a drug solution at high velocity in the form of a liquid microjet to allow the drug solution to directly penetrate into an internal target region through the skin's epidermis, instead of injecting a drug through the syringe needle.

The research of such a microjet drug delivery system was first attempted in the 1930s. The initial microjet drug delivery system is a very basic drug delivery method using a simple microjet mechanism. The above microjet drug delivery system involves various problems in that there is a risk of cross infection, a splash back phenomenon occurs during the microjet injection, and an accurate penetration depth is difficult to adjust. Particularly, since such a conventional microjet drug delivery system still has a disadvantage in that since the treatment is accompanied by a considerable pain, it was not widely adopted as an alternative to the conventional intracutaneous injection method.

In addition, as a method for addressing the pain-related problem involved in the above microjet drug delivery system and stabilizing the drug administration, Stachowiak et al. has developed and proposed a microjet drug delivery system using a piezoelectric ceramic element (J. C. Stachowiak et al, Journal of Controlled Release 124: 88-97 (2009)). The microjet drug delivery system proposed by Stachowiak et al. is one in which a drug is injected at high speed in the form of a liquid microjet using vibration generated when an electric signal is applied to the piezoelectric ceramic element. According to the microjet drug delivery system to Stachowiak et al., the drug can be stably injected intracutaneously into the skin without touching the nervous tissues through a real-time change in injection speed of the microjet, thereby effectively reducing a pain during the treatment. However, the microjet control of a trace amount of drug must be capable of being performed in order to implement the time-varying monitoring of the drug injection. The microjet drug delivery system using the piezoelectric ceramic element has a great difficulty in realizing an actual drug delivery system due to a limitation of microjet control precision.

Further, besides the above microjet drug delivery system using an electric element and device, according to a recent research result, it has been reported that a microjet drug delivery system using a laser was developed (V. Menezes, S. Kumar, and Takayama, Journal of Appl. Phys. 106, 086102 (2009)). Such a microjet drug delivery scheme using the laser is one in which a laser beam is irradiated onto an aluminum foil to generate a shock wave so that a drug solution is injected in the form of a microjet. The microjet drug delivery scheme has an advantage in that the laser permits high energy to be focused inside a very small area of the drug solution, enabling implementation of a precise level of needle-free drug delivery system. However, the above microjet drug delivery system using the laser beam and the shock wave entails problems in that continuously controlled microjet injection is impossible, and particularly the re-use of the used system is difficult because ablation occurs on the aluminum foil due to the irradiation of the laser beam thereto.

Thus, in order to solve the problems occurring in the microjet drug delivery system, the present inventor has developed a novel type of microjet drug delivery system that can inject a drug at high speed in the form of a microjet using liquid bubbles and an elastic membrane. Such a novel type of microjet drug delivery system was filed as Korean Patent Application No. 10-2010-0056637. An invention of a previously filed application by the present inventor employs a phenomenon that when strong energy such as a laser beam is concentrated to a liquid contained in a sealed chamber, bubbles are generated due to the optical breakdown of the molecular structure of the liquid. When the bubbles are generated in the liquid, the total volume of the chamber is increased to cause the elastic membrane defining one side of the chamber to be abruptly expanded outwardly to forcibly push the drug solution to the outside of the nozzle so that the microjet injection can occur.

However, the invention of a previously filed application by the present inventor entails a problem in that since a laser apparatus for generating bubbles by applying concentrated energy to a driving liquid contained in the chamber is additionally included as a main element in the drug delivery system, the manufacturing cost is greatly increased and the total volume of the chamber is increased, leading to decreased practicality.

Moreover, in an embodiment proposed in the invention of a previously filed application by the present inventor, a pressure chamber and an elastic membrane that are made of a rubber material was implemented as a single part. Thus, since there is a risk that the wall surface of the chamber will be melted by the heat produced upon the irradiation of a laser beam, strong energy cannot be used, making it difficult to obtain a sufficient microjet injection speed. In addition, the above invention of a previously filed application encounters a problem in that since some drug comes into contact with a side edge of the pressure chamber made of the rubber material in terms of the design property of the microjet drug delivery system, a rubber component flows into the drug by the heat of the laser, resulting in a risk that the rubber component will have an adverse effect on the human body. In addition, as a result of actually manufacturing and testing a prototype of the above invention of a previously filed application, it could be found that a complete sealing function was not attained due to its structure, resulting in leakage of waster at several points.

### DISCLOSURE OF INVENTION

### Technical Problem

Accordingly, the present invention has been made in order to solve the above-described problems occurring in the prior art, and it is a basic object of the present invention is to provide a microjet drug delivery system capable of substituting an conventional needle-type syringe and a microjet injector, in which a injection drug is sprayed at high speed in the form of a liquid microjet so as to penetrate into the skin tissue of the patient instead of injecting the drug into the tissues of the patient using a syringe needle, so that the drug solution can be effectively injected into the tissues of the patient in a more safe and convenient manner without any pain during the injection.

Another object of the present invention is to provide a novel type of microjet drug delivery system and an injector, in which strong energy such as a laser beam is concentrated to the inside of a liquid contained in a sealed chamber whose one side is partitioned by an elastic membrane to generate bubbles to cause abrupt expansion of the liquid volume to be transferred to the elastic membrane to allow a drug solution to be injected at high speed in the form of a microjet, so that a to-be-injected drug solution can be adjusted in the unit of a trace through the control of the amount of a laser beam irradiated, and thus a desired injection depth and penetration distribution can be controlled easily and the continuous supply of the drug solution and the repeated re-use of the injector are possible even after once injection, thereby effectively preventing waste of resources.

Still another object of the present invention is to provide a microjet drug delivery system and a microjet injector, which can be easily mounted to a medical laser apparatus widely used in an existing dermatology clinic or the like, so that the microjet drug delivery system can be easily implemented by utilizing an existing equipment through elimination of the necessity for purchasing an additional equipment and integrally forming the laser apparatus with the microjet injector.

Yet another object of the present invention is to improve a microjet drug delivery system which was filed previously by the present inventor and solve a problem of the invention of the previously filed application in that since the pressure chamber is made of the same rubber material as that of the elastic membrane, there is a risk that the chamber may be melted upon the irradiation of a laser beam and the rubber component may have an adverse effect on the human body, and a problem in that a effective sealing function was not attained due to its structure, resulting in leakage of waster.

### TECHNICAL SOLUTION

To achieve the above object, in one aspect, the present invention provides a microjet drug delivery system for spraying a drug solution stored therein so as to penetrate and to be injected into the human body or the animal body, the drug delivery system including: a microjet injector which comprises: a pressure chamber having a predetermined sealed accommodating space formed therein and configured to store a pressure-driving liquid in the sealed accommodating space; a pressure chamber having a predetermined accommodation space which is opened at one side thereof and configured to have a pressure-driving liquid hermetically filled in the accommodation space; an elastic membrane made of an elastic material and disposed so as to define a sealed space in the pressure chamber by closing the opened one side of the pressure chamber; a drug chamber disposed in proximity to the pressure chamber with the elastic membrane interposed between the pressure chamber and the drug chamber, and configured to accommodate a drug solution in a predetermined inner space; and a microjet nozzle fluidically communicating with the inner space of the drug chamber so as to be formed as a passageway for allowing the drug solution stored in the drug chamber to be injected to the outside therethrough; an energy focusing device configured to apply concentrated energy to the pressure-driving liquid stored in the pressure chamber to cause bubbles to be generated in the pressure-driving liquid; and a connecting adaptor configured to selectively detachably couple the microjet injector to the energy-focusing device.

In another aspect, the present invention provides a microjet injector including: a pressure chamber cylinder having a cylindrical shape which is internally hollow and is opened at both sides thereof; a transparent cap made of a transparent material to allows a laser beam emitted from the outside to pass therethrough, and disposed to close the opened one side of the pressure chamber cylinder; an elastic membrane made of an elastic material and configured to close the opened other side of the pressure chamber cylinder to define a sealed accommodating space in the pressure chamber cylinder; a pressure-driving liquid hermetically filled in the sealed accommodating space defined in the pressure chamber cylinder; and a nozzle block disposed in proximity to the pressure chamber cylinder with the elastic membrane interposed between the pressure chamber cylinder and the nozzle block, the nozzle block including a space defining a drug chamber for accommodating a drug solution therein and a microjet nozzle formed in fluid communication with one end of the drug chamber so as to allow the drug solution to be injected to the outside in the form of a microjet therethrough. In this microjet injector, the drug chamber is constructed so as to be partitioned at one side thereof by the elastic membrane so that when the elastic membrane is deformably expanded inward of the drug chamber by the generation of bubbles in the pressure-driving liquid, the drug solution can be injected to the outside through the microjet nozzle.

### Advantageous Effects

According to the drug delivery system of the present invention as constructed above, energy such as a laser beam is concentrated to a separate pressure-driving liquid, instead of directly applying an external force to a to-be-injected drug solution or performing another action in order to inject a drug solution, to induce the instantaneous generation of bubbles to cause the drug solution to be injected in the form of a microjet using the deformation of an elastic membrane due to the expansion of the liquid volume and the generation of a shock wave upon the generation and collapse of the bubbles, so that the drug solution can effectively penetrate in vivo without any pain during the injection

In addition, according to the microjet injector of the present invention, the microjet injector can be easily mounted to a medical laser apparatus widely used in an existing dermatology clinic or the like, so that the microjet injector can be manufactured and distributed in the form of a compact basic structure even without providing a separate energy supply means needed for the microjet injection, and thus it is expected that applicability and practicability will be very excellent and the manufacturing cost will be greatly reduced.

Further, according to the microjet injector of the present invention, the pressure chamber is formed in a cylindrical shape and a separate elastic membrane is coupled to one side of the cylindrical pressure chamber, so that strong laser beam can be irradiated onto a pressure-driving liquid, enabling efficient microjet injection.

Moreover, according to a preferred embodiment of the microjet injector of the present invention, respective constituent elements are separated and assembled according to each part, so that the microjet injector can be easily manufactured and a structurally complete sealing function can be attained, thereby preventing a leakage of water.

Furthermore, according to the present invention, the continuous supply of the drug solution and the repeated re-use of the injector are possible even after once injection, thereby preventing waste of resources according to the use of an existing disposable needle-type syringe. In addition, the microjet injector according to the present invention has an advantage in that a microjet injection is automatically performed by the operation of the laser apparatus unlike an existing needle-type syringe scheme in which an operator directly pierces the skin of the subject, thereby enabling an accurate injection without a risk of an error by a manual medical procedure.

Besides the above-mentioned effects, the present invention has various merits and advantageous effects, and the additional effects and merits of the present invention will be more apparent through the following description of the preferred embodiments.

### BRIEF DESCRIPTION OF THE DRAWINGS

The above as well as the other objects, features and advantages of the present invention will be more apparent from the following detailed description taken in conjunction with the accompanying drawings, in which:

FIG. 1 is a perspective view illustrating a mechanism in which a drug solution is injected in the form of a microjet in a microjet drug delivery system in accordance with the present invention;

FIG. 2 illustrates photographs taken on a series of processes in which a microjet injection is performed using a microjet injector that is trial-manufactured and is actually operated in accordance with the present invention.

FIG. 3 is an exploded perspective view illustrating the construction of a microjet injector and a connecting adaptor in accordance with a preferred embodiment of the present invention;

FIG. 4 is an assembled cross-sectional view illustrating the microjet injector and the connecting adaptor shown in FIG. 3;

FIG. 5 is a simplified cross-sectional view illustrating the microjet injector shown in FIG. 4 to further emphasize the inner space arrangement of the microjet injector of the present invention;

FIG. 6 is a perspective view illustrating a preferred use state of a microjet injector of the present invention in which the microjet injector is mounted at a medical therapy laser; and

FIG. 7 illustrates photographs taken on the result of a test performed on the adipose tissue of pork using a microjet injector manufactured in accordance with a preferred embodiment of the present invention.

*Explanation on reference numerals of main elements in
the drawings*

| 1: inventive microjet injector | | |
|---|---|---|
| 2: energy-focusing device | 3: | laser handpiece |
| 10: pressure chamber | 12: | pressure chamber cylinder |
| 20: drug chamber | 22: | nozzle block |
| 25: microjet nozzle | 30: | elastic membrane |
| 40: transparent cap | 35, 45: | rubber packing |
| 50: cap holder | 55: | adaptor-coupling unit |
| 56: retaining step | 60: | elastic membrane holder |
| 65: drug supply passage | 70: | nozzle holder |
| 78: drug tube hole | 79: | drug supply tube |
| 80: connecting adaptor | 85: | retaining ring |
| 100: pressure-driving liquid | 200: | drug solution |

### BEST MODE FOR CARRYING OUT THE INVENTION

Hereinafter, the mechanism and technical concept of the present invention will be described with reference to the accompanying drawings, and the present invention will be described in further detail by way of embodiments in which this technical concept of the present invention is implemented in a preferred manner.

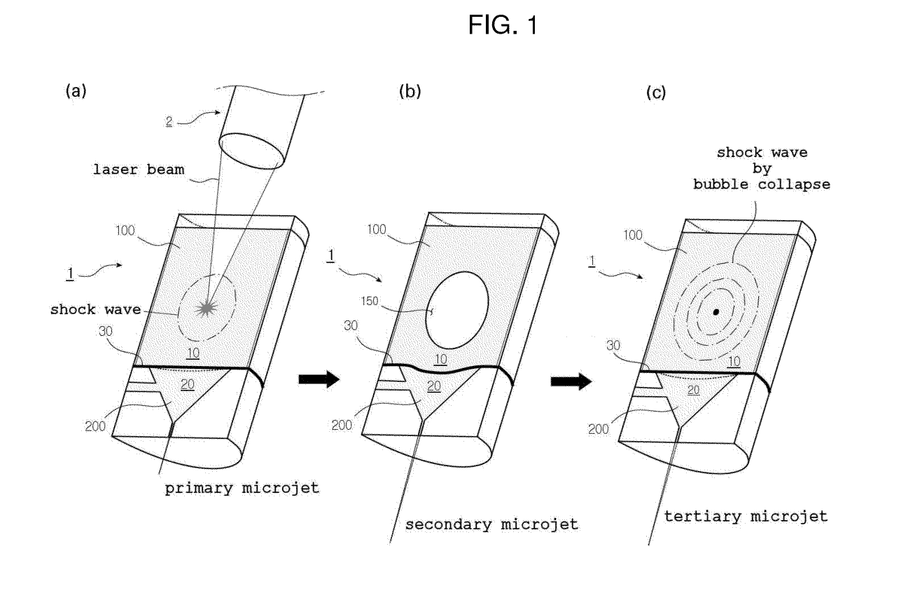

FIG. 1 is a perspective view illustrating a mechanism in which a drug solution is injected in the form of a microjet in a microjet drug delivery system in accordance with the present invention.

As shown in FIGS. 1(a) to 1(c), the microjet drug delivery system according to the present invention roughly includes a microjet injector 1 serving as an injection device that stores a predetermined amount of a drug solution therein and injects the drug solution to the outside in the form of a microjet so as to be administered in vivo, and an energy-focusing device 2 serving as a means for supply driving energy required to inject the drug from the microjet injector 1.

The microjet injector 1 is configured such that two chambers are successively formed in a single housing. That is, a drug chamber 20 for storing a drug solution to be injected (also called “to-be-injected drug solution”) is disposed at a front side of the microjet injector 1, and a pressure chamber 10 internally filled with a pressure-driving liquid 100 is disposed at a rear side of the microjet injector 1. The pressure chamber 20 corresponds to a pressure chamber for applying a driving force to the drug solution 200 stored in the drug chamber 20. In addition, a boundary wall that divides the drug chamber 20 and the pressure chamber 10 is formed as a membrane made of an elastic material, so that the boundary wall is expanded and deformed elastically depending on the physical state change of the pressure-driving liquid 100 contained in the pressure chamber 10 to cause pressure to be applied to the drug solution 200 stored in the drug chamber 20.

In the inventive microjet injector 1 as constructed above, the driving force for injecting the drug solution 200 in the form of a microjet is generated from the pressure-driving liquid 100 filled in the pressure chamber 10. In the present invention, bubbles are generated within the pressure-driving liquid 100 hermetically filled in the pressure chamber 10 and the elastic membrane 30 is instantaneously strongly pushed toward the drug chamber 20 by an increase in the entire volume or a transfer of a shock wave due to the generation of the bubbles so that a driving pressure is applied to a to-be-injected drug solution 200 contained in the drug chamber 20.

In other words, as shown in FIG. 1, when strong energy (for example, laser beam or electric spark) is instantaneously concentrated to the pressure-driving liquid 100 hermetically filled in the pressure chamber 10 of the present invention, the pressure-driving liquid receives the concentrated energy and optical breakdown occurs in its molecular structure to cause bubbles to be generated in the pressure-driving liquid. In this case, the bubbles are expanded instantaneously and then are collapsed immediately when the irradiation of a laser beam is stopped. The elastic membrane is expanded and deformed outwardly (i.e., in the direction of the drug chamber) by the shock wave generated upon the abrupt expansion and generation/collapse of the bubbles. This deformation of the elastic membrane acts as an external force to the drug solution 200 contained the drug chamber 20 so that the drug solution is injected in the form of a microjet at high speed enough to penetrate into the skin tissue of a subject through a microjet nozzle 25 having a very small diameter.

Referring to FIG. 1, the above microjet generation mechanism of the present invention will be described in more detail sequentially over time.

For example, the case where a laser irradiation device is used as the energy-focusing device that supplies energy needed to generate the bubbles will be described. First, as shown FIG. 1(a), a laser beam emitted from a laser unit 2 is focusably irradiated onto the pressure-driving liquid 100 contained in the sealed pressure chamber 10, some liquid at a focal point in the pressure-driving liquid 100 receive the concentrated energy of the laser beam and the optical breakdown occurs in its molecular structure. The shock wave caused by the optical breakdown occurring in the molecular structure of the pressure-driving liquid upon the irradiation of the laser beam is transferred to the elastic membrane 30 to cause micro vibration to occur on the elastic membrane, and the drug solution 200 contained in the drug chamber 20 receives pressure due to the vibration of the elastic membrane 30 so that a primary microjet injection of a relatively low rate (25 m/s or so) occurs.

In addition, as a result of the optical breakdown occurring upon the irradiation of the laser beam, as shown in FIG. 1(b), the pressure-driving liquid 100 is vaporized to generate vapor bubbles. Thereafter, the generated vapor bubbles are expanded abruptly and then are collapsed. The elastic membrane 30 is pushed outwardly and elastically expanded rapidly due to the abrupt expansion of the bubbles. As a consequence, the drug solution contained in the drug chamber adjacent to the elastic membrane 30 is strongly pushed instantaneously and is pressurized so that a secondary microjet injection occurs. This secondary microjet exhibits a relatively high rate of 230 m/s or so as compared to that of the primary microjet, and allows the drug solution to be injected in a larger amount than that in the primary microjet.

A tertiary microjet injection occurs immediately after the occurrence of the secondary microjet injection. The tertiary microjet injection occurs by the shock wave according to the collapse of the bubbles. The bubbles 150 are maintained for a short time immediately after being generated in the pressure-driving liquid 100, and then are collapsed immediately. Along with the collapse of the bubbles, the pressure-driving liquid 100 is abruptly contracted to its original state to generate a secondary shock wave to cause the elastic membrane 30 to be strongly vibrated rapidly to push the drug solution 200 so as to be sprayed to the outside. As can be seen from the experimental result which will be described later, the largest amount of microjet is injected at the highest rate due to the transfer of the secondary shock wave generated by the collapse of the bubbles

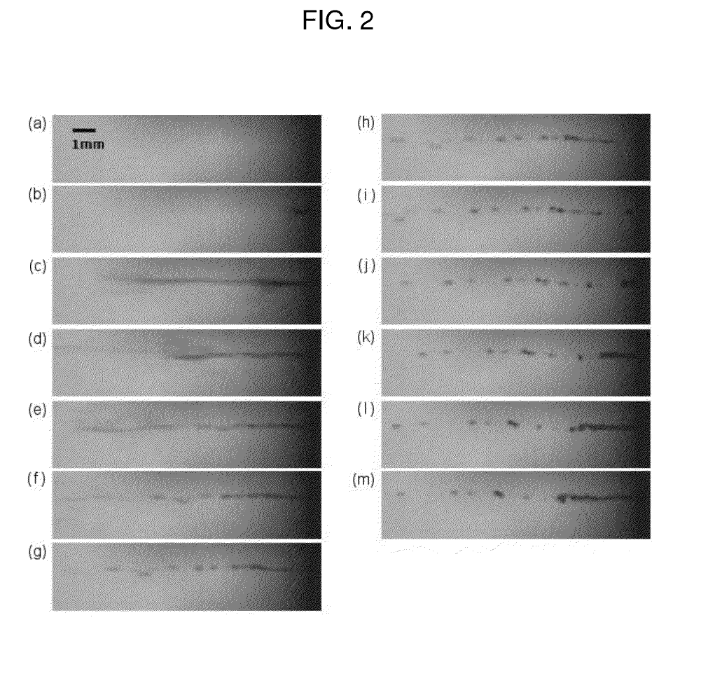

FIG. 2 illustrates photographs taken on a series of processes in which a microjet injection is performed using a microjet injector that is trial-manufactured and is actually operated in accordance with the present invention.

The series of processes were photographed by a 75000 fps very high speed camera as the photographing equipment. In the photographs of FIG. 2, the time interval is 40 ms. A laser equipment used in the experiment was a Q-Switched Nd:YAG medical laser (model name: Spectra Laser Platform) manufactured and sold by Lutronic Corporation in the US. The medical laser irradiated a laser beam onto the pressure-driving liquid in the pressure chamber with a wavelength of 1064 nm, a pulse energy of 314 mJ and a pulse interval of 507 ns. The diameter of the microjet nozzle was set to 0.1 mm and the diameter of the microjet was observed to be about 0.1 mm.

As an experimental result, as shown in FIG. 2, it could be found that three microjets are injected. Is can be found from a second photograph (see FIG. 2(b)) among the successive photographs in FIG. 2 that a first microjet is generated at a low rate of less than 25 m/s. It can be found from a photograph of FIG. 2(c) that a second microjet is generated at a very high rate of more than 230 m/s and passes through the previously generated first jet. Also, it can be found from a photograph of FIG. 2(h) that a third microjet is injected at the largest amount among the three jets.

Briefly, it can be seen that when a laser beam is irradiated onto the microjet injector according to the present invention, the following three microjets are generated to be injected: a primary microjet generated by the transfer of the primary shock wave caused by the optical breakdown occurring in the molecular structure of the pressure-driving liquid upon the irradiation of the laser beam, a secondary microjet generated by the expansion of the bubbles, and a tertiary microjet generated the transfer of the secondary shock wave caused by the collapse of the bubbles.

Thus, in the case where the microjet injector according to the present invention is actually used in the human body or the animal body, a relatively weak preliminary shock can be applied to the human or animal skin by injection of the low-speed primary microjet at an early stage to alleviate the pain felt during the injection through the nerve disturbance. Next, an injury is caused on the epidermal tissue of the skin by injection of the high-speed secondary microjet to perforate the skin so that a drug solution can be administered in vivo. Then, a large amount of drug solution is sprayed by injection of the tertiary microjet so as to be administered into the skin tissue. Therefore, it can be expected that the delivery of a drug will be carried out by the above drug administration method.

Hereinafter, a preferred embodiment that can implement and carry out the technical concept of the present invention will be proposed and described in detail.

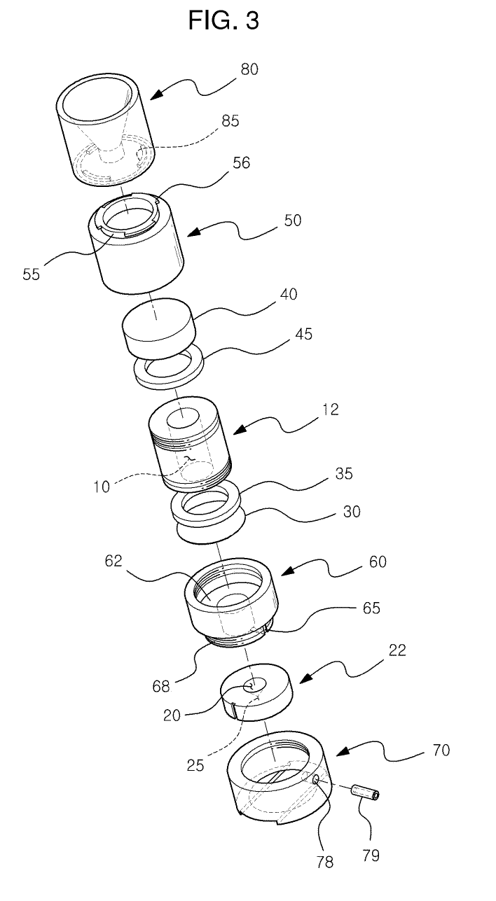

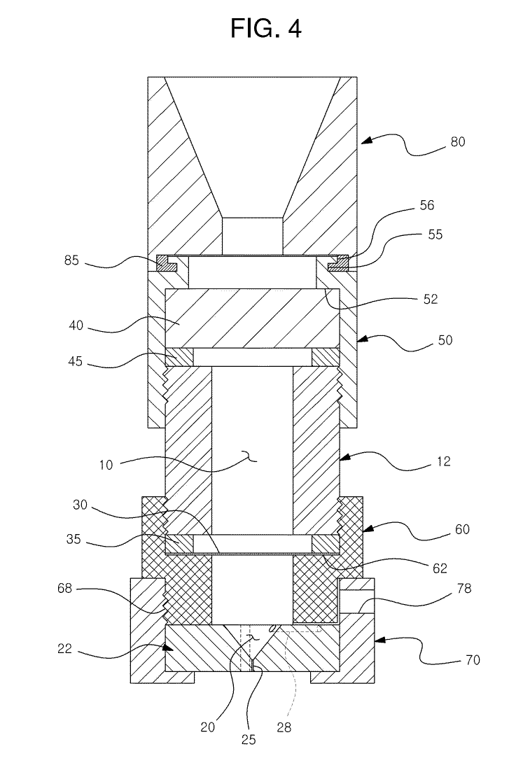

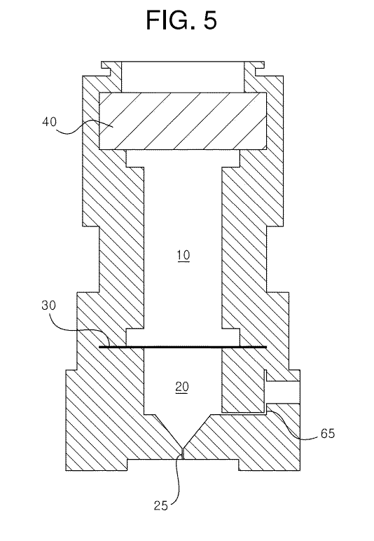

FIG. 3 is an exploded perspective view illustrating the construction of a microjet injector and a connecting adaptor in accordance with a preferred embodiment in which the basic technical construction of the present invention shown in FIG. 1 is implemented in the preferred form that can be actually carried out, and FIGS. 4 and 5 are an assembled and simplified cross-sectional view illustrating the microjet injector and the connecting adaptor shown in FIG. 3.

As can be seen in the embodiment shown in FIGS. 3 to 5, a microjet injector 1 used in the microjet drug delivery system basically includes a pressure chamber 10 configured to store a pressure-driving liquid 100 in a predetermined sealed accommodation space formed therein; a drug chamber 20 disposed in proximity to the pressure chamber, configured to accommodate a to-be-injected drug solution in a predetermined inner space, and having one side at which a microjet nozzle is formed; and an elastic membrane configured to divide the pressure chamber 10 and the drug chamber 20.

According to the embodiment shown in FIGS. 3 to 5, in actually constituting the pressure chamber 10, the drug chamber 20, the elastic membrane 30, and the microjet nozzle 25, which are essential elements for implementing the technical concept of the present invention, the above essential elements are manufactured using parts such as a transparent cap 40, a pressure chamber cylinder 12, the elastic membrane 30, and a nozzle block 22. In addition, these respective parts are assembled by being coupled with each other sequentially. In this case, in a state in which a rubber packing 45 is disposed between the parts to seal the gaps defined between the parts, a ring screw type cap holder 50, an elastic membrane holder 60, and a nozzle holder 70 are fastened together so that the microjet injector 1 is assembled.

Meanwhile, although a laser apparatus for irradiating a laser beam is not specifically shown in FIGS. 3 to 5, it can use an Nd:YAG medical laser equipment or the like that is widely known in the art (e.g., in the dermatological field). In the present invention, a connecting adaptor 80 is further provided to couple the microjet injector to the laser equipment.

The respective constituent elements constituting the microjet injector 1 of the present invention shown in FIGS. 3 to 5 will be described hereinafter in further detail.

In this embodiment, the pressure chamber 10 is implemented using a pressure chamber cylinder 12 having a cylindrical shape which is internally hollow and is opened at both sides thereof. The opened one side of the pressure chamber cylinder 12 is closed by the transparent cap 40 and the opened other side of the pressure chamber cylinder 12 is closed by the elastic membrane 30, which will be described later, so that the pressure chamber cylinder 12 internally defines a sealed accommodating space in its entirety. In this embodiment, the pressure chamber cylinder 12 is made of a stainless steel strongly resistant to heat so that it can endure the heat upon the irradiation of the laser beam. Besides, a person of ordinary skill in the art may select various materials and may manufacture the pressure chamber cylinder using the various materials as long as different kind of a single metal or a metal alloy, a synthetic resin material, or the like can perform the function of the present invention without any difficulty.

The transparent cap 40 serving to close the opened one side of the pressure chamber cylinder 12 is made of a transparent material to allow a laser beam emitted from the outside to pass therethrough so that the laser beam can be focused on the inside of the pressure-driving liquid 100 contained in the pressure chamber 10. The transparent cap 40 is preferably made of a BK7 glass material, but may be made of another glass material or a transparent plastic material. In addition, the transparent cap 40 may have the shape of a convex lens (not shown) which is bulged at the central portion thereof so as to allow the laser beam passing through the transparent cap to be converged to cause stronger energy to be concentrated on the pressure-driving liquid 100.

The cap holder 50 is a fixing part used to couple the transparent cap 40 to the pressure chamber cylinder 12. As shown in FIGS. 3 to 5, the cap holder 50 is a ring screw member that is centrally hollow in its entirety and has a screw thread formed on the inner peripheral surface thereof. The inner diameter of an opening of the bottom of the cap holder 50 is sized to correspond to the outer diameter of the pressure chamber cylinder 12. A cap retaining step 52 is formed at an opening of the top of the cap holder 50 so that the top circumferential edge of the transparent cap 40 can be fixedly retained on the underside of the cap retaining step 52 by being pressed by the cap retaining step 52. Thus, in a state in which the transparent cap 40 is placed on the pressure chamber cylinder 12, when the cap holder 50 is turned with it fitted around the top of the pressure chamber cylinder 12, the cap holder 50 is tightly engaged with the pressure chamber cylinder 12 to cause the transparent cap 40 to be pressed against the top of the pressure chamber cylinder 12 so that the pressure chamber cylinder 12, the transparent cap 40, and the cap holder 50 are integrally coupled with one another. In this case, a ring type rubber packing 45 cam be additionally provided between the transparent cap 40 and the pressure chamber cylinder 12 to provide a sealing function.

Moreover, according to a preferred aspect of the present invention, the cap holder 50 is constructed so as to have an adaptor-coupling unit 55 formed on the top thereof to couple the connecting adaptor 80, which will be described later, to the cap holder 50. The connecting adaptor 80, which will be described later, is a part for detachably coupling the microjet injector 1 of the present invention to an adaptor for an external apparatus so as to be engaged to a front end tip a standard handpiece 3 of an existing medical laser apparatus.

An elastic membrane 30 is disposed at the opened other side (i.e., the bottom in the drawings) of the pressure chamber cylinder 12 so that the both sides of the pressure chamber cylinder 12 are closed by the transparent cap 40 and the elastic membrane 30 to define the pressure chamber 10 as the sealed accommodating space within the pressure chamber cylinder 12. The elastic membrane 30 is a thin film member made of an elastic material such as natural or synthetic rubber. The elastic membrane 30 has such physical properties that it is maintained in a state of being stretched tightly and then is deformable and restorable elastically when receiving physical pressure from the outside. The elastic membrane 30 is preferably made of a nitril butadiene rubber (NBR) material that has a thickness of 200 μm, a hardness of 53, an ultimate strength of 101.39 kg/cm², and an elongation of 449.79%. The NBR material has an excellent flexibility as well as a low thermal conductivity, and thus can prevent damage of the drug solution due to the heat transfer upon the irradiation of a laser beam.

The elastic membrane 30 can be coupled to the pressure chamber cylinder 12 using a ring screw type elastic membrane holder 60 in a similar manner to that described when the transparent cap 40 is coupled to the pressure chamber cylinder 12. As shown in FIG. 3, in a state in which the elastic membrane 30 is positioned beneath of the underside of the pressure chamber cylinder 12, when the elastic membrane holder 60 is turned with it fitted around the bottom of the pressure chamber cylinder 12 while surrounding the elastic membrane 30, the elastic membrane holder 60 is tightly engaged with the pressure chamber cylinder 12 to cause a pressing step 62 formed on the inner peripheral surface of the elastic membrane holder 60 to press the elastic membrane 30 so that the elastic membrane 30 is firmly coupled to the pressure chamber cylinder 12. In this case, a ring type rubber packing 35 cam be additionally provided between the elastic membrane 30 and the pressure chamber cylinder 12 to provide a sealing function as described above.

A pressure-driving liquid 100 is hermetically filled in the pressure chamber 10 defined by the pressure chamber cylinder 12, the transparent cap 40, and the elastic membrane 30. The pressure-driving liquid 100 is intended such that when it receives very strongly concentrated energy like the laser beam as in the aforementioned mechanism of the present invention, the optical breakdown occurring in its molecular structure to cause bubbles to be produced in the pressure-driving liquid so as to provide a driving force required for the drug solution to be injected in the form of a microjet. The pressure-driving liquid 100 may be a liquid material, sol, gel, or the like that can absorb energy from a laser apparatus or an electric spark to generate bubbles. That is, in the present invention, examples of the pressure-driving liquid 100 include all kinds of fluidable liquid materials such as a single liquid component such as water or alcohol, a mixture of two or more liquid components, and sol or gel prepared by mixing a liquid with a solid.

In this embodiment, degassed water was used as the pressure-driving liquid 100 to minimize residual bubbles before and after the irradiation of a laser beam and the injection of the drug. Various liquid materials including polymeric sol or gel such as other alcohol or polyethylene glycol, or the like may be used as the pressure-driving liquid 100. In addition, when aqueous electrolyte (e.g., salt) is added to pure water as the pressure-driving liquid 100, energy required for the optical breakdown of the liquid becomes small due to the ionization of the water molecules, and thus the efficiency can be improved so much.

At one side of the pressure chamber 10 as constructed above with respect to the elastic membrane 30, is successively formed the drug chamber 20 as another main constituent element for implementing the drug delivery system of the present invention. The drug chamber 20 is a predetermined space portion that stores a to-be-injected drug solution 200 therein. The drug chamber 20 is constructed such that the elastic membrane 30 is disposed at one side thereof and a microjet nozzle 25 is provided at the other side thereof to serve as a passageway allowing the drug solution 200 to be injected to the outside therethrough so that the drug solution 200 can be injected in the form of a microjet by the elastic deformation of the elastic membrane 30.

In the meantime, in the present invention, the drug solution 200 refers to all kinds of injection drugs that can be administered in vivo through the microjet injector of the present invention. Examples of this drug solution include a variety of kinds of drug solutions such as cosmetic emulsions (e.g. hyaluronic acid (HA) solution, HA filler, retinol, etc.), anesthetics, hormone drugs, preventive vaccines, mesotherapy drugs (e.g. adipolytic agents), and the like, including various drugs for treatment.

The microjet nozzle 25 is an opening with a very minute cross-section diameter, which is provided at a front end of the nozzle block 22 so that when the drug solution is pressed so as to be sprayed from the drug chamber 20, it can be injected in the form of a microjet at high speed under high pressure. The microjet nozzle 25 can be implemented in the form of a single pore but may be implemented in the form of two or more multi-pores. Like this, in the case where the microjet nozzle 25 is implemented in the form of the multi-pores, the drug administration area per each injection can be increased and the same effect as in the skin patch can be exhibited.

In the embodiment shown in FIGS. 3 to 5, the drug chamber 20 is implemented by the elastic membrane 30, a part (i.e., the inner space of a lower end side) of the elastic membrane holder 60, and the nozzle block 22 which will be described later. According to this embodiment, the nozzle block 22 is provided in the form of a single part that includes a microjet nozzle 25 formed at one side thereof, is opened at the other side thereof, and internally defines an accommodating space having a predetermined volume. The accommodating space for defining the drug chamber 20 provided in the nozzle block 22 is formed in a conical shape whose diameter is reduced as it goes toward the microjet nozzle 25 in its entirety as shown FIGS. 4 and 5. By virtue of this construction, when the drug solution 200 is pressed by the deformable expansion of the elastic membrane 30, the drug solution can be strongly injected to the outside efficiently in the form of a microjet without any distribution of pressure.

In this case, more preferably, when the microjet nozzle is coated on the inner peripheral surface thereof with polytetrafluoroethylene (known by trademark name Teflon®) or the like, the friction coefficient and the surface tension between the nozzle surface and the drug solution becomes low so that the injection of the drug solution can be further performed, thereby contributing to the efficiency improvement of the microjet injector.

The nozzle block 22 is assembled such that it is coupled to the bottom of the elastic membrane holder 60 in a state in which the opened side of the inner accommodating space thereof is oriented toward the elastic membrane 30. Preferably, a method using a ring screw similar to the coupling method between elements as described mentioned can be applied to the coupling of the nozzle block 22 to the elastic membrane holder 60. In other words, as shown in FIG. 3, a nozzle holder 70 for securely fixing the nozzle block 22 is provided in the form a cylindrical ring screw that has a screw thread formed on the inner peripheral surface thereof. In addition, the depth and inner diameter of the nozzle holder 70 is sized such that a screw thread formed on the inner peripheral surface of the nozzle holder 70 is screwably engaged to a screw thread 68 formed on the outer peripheral surface of the a stepped lower end of the elastic membrane holder 60 in a stated in which the nozzle block 22 is completely inserted into the nozzle holder 70. Thus, in a state in which the nozzle block 22 is positioned beneath the underside of the elastic membrane holder 60, when and the nozzle holder 70 is turned with it fitted around the bottom of the elastic membrane holder 60 while surrounding the nozzle block 22, the nozzle holder 70 is tightly engaged with the elastic membrane holder 60 to cause the nozzle block 22 to be pressed against the elastic membrane holder 60 so that the nozzle block 22 can be firmly coupled to the elastic membrane holder 60.

Meanwhile, according to the embodiment shown in FIGS. 3 to 5, the elastic membrane holder 60 further includes a drug supply passage 65 formed at a side thereof so that an additional drug can be continuously supplied to the inside of the drug chamber 20. The drug supply passage 65 is connected to a separate external drug supply unit (not shown) that stores a large amount of drug so that an additional drug can be supplied to the drug chamber 20 immediately after the drug solution contained in the drug chamber 20 is injected to the outside in the form of a microjet. The external drug supply unit can be implemented in various manners depending on the design of a person of ordinary skill in the art. Basically, the drug supply unit is designed such that it includes a given pressure means so that when the drug chamber 20 is empty, a drug can be re-supplied to the drug chamber through suitable pressure.

In the meantime, the nozzle holder 70 further includes a drug tube hole 78 formed at a side thereof so as to be connected to the drug supply passage 65 of the elastic membrane holder 60 to define a drug supply channel. After the drug supply tube 79 connected to the drug supply unit is inserted into the drug tube hole 78 and then the drug solution is injected, a drug solution is additionally supplied into the drug chamber 20. In this embodiment shown in FIGS. 3 to 5, although it is illustrated that the drug supply passage 65 is formed in the elastic membrane holder 60, it may be provided at a side or another portion of the nozzle block 22 in such a manner as to fluidically communicate with the drug chamber 20.

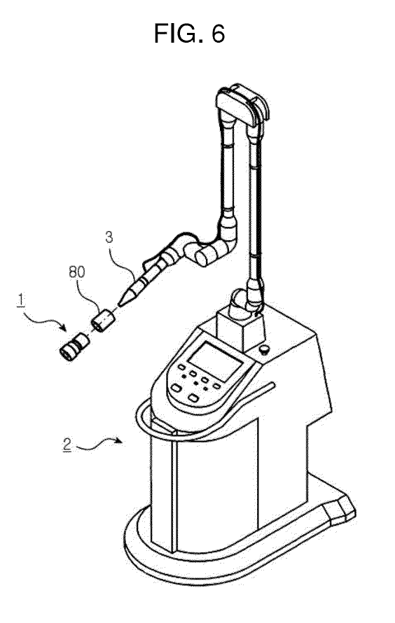

Next, according to another feature of the present invention, a connecting adaptor 80 is additionally coupled to the microjet injector 1 of the present invention so as to be coupled to an external apparatus. The connecting adaptor 80 is intended to couple the microjet injector 1 of the present invention to the energy-focusing device as one of the main elements constituting the microjet drug delivery system of the present invention. In the present invention, the connecting adaptor 80 is constructed such that it is mounted to a front end tip a handpiece 3 of a laser apparatus and is again separated from the tip of the handpiece 3 as shown in FIG. 6.

Thus, according to the present invention, since the connecting adaptor 80 is separately included in the microjet drug delivery system of the present invention, a module capable of applying strong energy to the pressure-driving liquid 100 contained in the above-mentioned pressure chamber 10 does not need to be integrally formed with the microjet injector. The microjet injector 1 and the connecting adaptor 80 may be simply manufactured, distributed, and carried, and may be easily used by being mounted on a medical laser apparatus equipped in a hospital or the like.

FIG. 6 is a perspective view illustrating a preferred use state of a microjet injector of the present invention in which the microjet injector is mounted at a medical therapy laser.

In particular, as a result of the experiment performed by the present invention, it is determined that a laser is the most suitable for an energy supply source used in the drug delivery system of the present invention. Currently, a Q-switched Nd:YAG laser is widely supplied and used as a medical laser apparatus in a dermatology clinic, a dental clinic, and the like. Accordingly, if the connecting adaptor for the microjet injector 1 of the present invention is provided in the form that can be mounted to a front end tip the handpiece 3 of the above existing medical laser apparatus, it can be expected that utilization of the present invention will be able to be further increased.

As shown in FIGS. 3 and 4, the connecting adaptor 80 is connected at one side thereof to the cap holder 50 of the microjet injector 1 of the present invention as mentioned above and is fixedly connected at the other side thereof to the tip of the handpiece of the medical laser apparatus. In addition, according to the embodiment shown in the drawings, the cap holder 50 includes an adaptor-coupling unit 55 having a retaining step 56 formed protrudingly outwardly therefrom in a segment shape so that the cap holder 50 can be detachably coupled to the connecting adaptor 80. Also, the connecting adaptor 80 includes a retaining ring 85 formed on the inner peripheral surface of the bottom thereof so as to correspond to the retaining step 56 so that the retaining step 56 of the adaptor-coupling unit 55 can be fittingly engaged with the retaining ring 85. Thus, when the retaining step 56 protruded from adaptor-coupling unit 55 is inserted into a space portion of the retaining ring 85 of the connecting adaptor 80 and then the cap holder 50 is axially rotated while being turned by a predetermined angle, the retaining step 56 is positioned behind the protruded portion of the retaining ring 85 so that the cap holder 50 can be easily fixedly coupled to the connecting adaptor 80 and can be easily removed from the connecting adaptor 80.

Further, the other side of the connecting adaptor 80 is machined and manufactured to conform to the shape of the tip of the handpiece of the medical laser apparatus so that the connecting adaptor 80 can be coupled to and decoupled from the tip of the handpiece. The connecting adaptor 80 is manufactured to have a proper length such that a focal point of a laser beam emitted from the laser apparatus is converged to the pressure-driving liquid 100 contained in the pressure chamber 10. Although not shown, a separate objective lens may be additionally included in the connecting adaptor 80 or the transparent cap 40 may be machined in the shape of a convex lens to serve as the objective lens in order to adjust a focal distance.

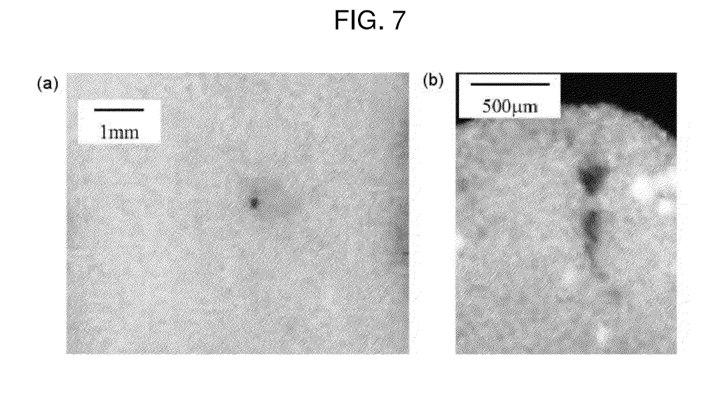

FIG. 7 illustrates photographs taken on the result of a test performed on the adipose tissue of pork using a microjet injector manufactured in accordance with a preferred embodiment of the present invention shown in FIGS. 3 to 5.

In this experiment, the diameter of the microjet nozzle of the microjet injector was set to 100 μm, a laser beam was irradiated onto the pressure-driving liquid in the pressure chamber with a wavelength of 1064 nm, a pulse energy of 3 J and a pulse interval of 5 to 10 ns using a spectra laser platform as a Q-Switched Nd:YAG medical laser apparatus available from Lutronic Corporation in the US. A black aqueous ink was used as a to-be-injected liquid

In FIG. 7, FIG. 7(a) is a photograph taken from the top of the adipose tissue of pork into which the drug solution penetrates, and FIG. 7(b) is a photograph taken from the side of adipose tissue of pork which is frozen in a freezer after the penetration of the drug solution and then is the penetrated region is cut off by a knife. It can be found experimentally from the result of FIG. 7 that the diameter of the hole formed in the penetrated adipose tissue was about 0.15 mm, and the depth of the hole formed in the penetrated adipose tissue about 0.75 mm, so that the microjet injector of the present invention can perforate the skin tissue to allow the drug solution to be administered in vivo.

### INDUSTRIAL APPLICABILITY

Therefore, according to the microjet injector and the drug delivery system provided by the present invention, various kinds of drug solutions such as a variety of drugs for treatment, cosmetic emulsions, anesthetics, hormone drugs, vaccines, and the like can be rapidly administered into the human body or the animal body without feeling pain during injection, and thus the present invention is expected to be desirably utilized in various fields such as a medical field, a cosmetic field, a livestock field, etc.

1. A microjet drug delivery system, comprising:
a pressure chamber having a predetermined sealed accommodating space formed therein and configured to store a pressure-driving liquid in the sealed accommodating space;
a microjet injector which comprises: a pressure chamber having a predetermined accommodation space which is opened at one side thereof and configured to have a pressure-driving liquid hermetically filled in the accommodation space; an elastic membrane made of an elastic material and disposed so as to define a sealed space in the pressure chamber by closing the opened one side of the pressure chamber; a drug chamber disposed in proximity to the pressure chamber with the elastic membrane interposed between the pressure chamber and the drug chamber, and configured to accommodate a drug solution in a predetermined inner space;
and a microjet nozzle fluidically communicating with the inner space of the drug chamber so as to be formed as a passageway for allowing the drug solution stored in the drug chamber to be injected to the outside therethrough;
an energy focusing device configured to apply concentrated energy to the pressure-driving liquid stored in the pressure chamber to cause bubbles to be generated in the pressure-driving liquid; and
a connecting adaptor configured to selectively detachably couple the microjet injector to the energy-focusing device.

2. The microjet drug delivery system according to claim 1, wherein the energy-focusing device is a laser generator that generates a laser beam and focusably irradiates the generated laser beam onto the pressure-driving liquid stored in the pressure chamber.

3. The microjet drug delivery system according to claim 2, wherein the energy-focusing device is a medical laser apparatus used for medical purpose, and the connecting adaptor is coupled to a front end tip of a handpiece included in the medical laser apparatus.

4. The microjet drug delivery system according to claim 3, wherein the medical laser apparatus is an Nd:YAG laser.

5. The microjet drug delivery system according to claim 1, wherein the pressure-driving liquid comprises an electrolyte dissolved therein.

6. The microjet drug delivery system according to claim 1, wherein the drug chamber is connected to a drug reservoir having a drug solution contained therein so that the drug solution stored in the drug chamber can be injected to the outside in the form of a microjet and then can be supplied with the drug solution from the drug reservoir.

7. The microjet drug delivery system according to claim 1, wherein the drug chamber has an inner accommodating space formed in a conical shape whose diameter is reduced as it goes toward the microjet nozzle.

8. The microjet drug delivery system according to claim 1, wherein the microjet nozzle is further formed on the inner peripheral surface thereof with a coating layer.

9. The microjet drug delivery system according to claim 2, wherein one side of the pressure chamber is composed of a plate made of a transparent material to allow the laser beam to pass therethrough.

10. The microjet drug delivery system according to claim 9, wherein the transparent material is BK7 glass.

11. A microjet injector comprising:
a pressure chamber cylinder having a cylindrical shape which is internally hollow and is opened at both sides thereof;
a transparent cap made of a transparent material to allows a laser beam emitted from the outside to pass therethrough, and disposed to close the opened one side of the pressure chamber cylinder;
an elastic membrane made of an elastic material and configured to close the opened other side of the pressure chamber cylinder to define a sealed accommodating space in the pressure chamber cylinder;
a pressure-driving liquid hermetically filled in the sealed accommodating space defined in the pressure chamber cylinder; and
a nozzle block disposed in proximity to the pressure chamber cylinder with the elastic membrane interposed between the pressure chamber cylinder and the nozzle block, the nozzle block including a space defining a drug chamber for accommodating a drug solution therein and a microjet nozzle formed in fluid communication with one end of the drug chamber so as to allow the drug solution to be injected to the outside in the form of a microjet therethrough,
wherein the drug chamber is constructed so as to be partitioned at one side thereof by the elastic membrane so that when the elastic membrane is deformably expanded inward of the drug chamber by the generation of bubbles in the pressure-driving liquid, the drug solution can be injected to the outside through the microjet nozzle.

12. The microjet injector according to claim 11, wherein a connecting adaptor is further provided at one side of the microjet injector so that the microjet injector can be detachably mounted to an external laser apparatus that irradiates a laser beam onto the pressure-driving liquid to generate bubbles in the pressure-driving liquid.

13. The microjet injector according to claim 11, wherein the pressure chamber cylinder is made of a metal or synthetic resin material and the elastic membrane is made of an elastic rubber material.

14. The microjet injector according to claim 12, wherein the connecting adaptor is implemented in the form that can be coupled to a front end tip of a handpiece included in a medical laser apparatus.

15. The microjet injector according to claim 11, wherein the transparent cap and the pressure chamber cylinder are engaged with each other such that the transparent cap is disposed on the opened one side of the pressure chamber cylinder and a ring screw type cap holder is engagingly fitted around the top of the pressure chamber cylinder so as to surround the transparent cap.

16. The microjet injector according to claim 15, further comprising a ring-shaped rubber packing interposed between the transparent cap and the pressure chamber cylinder.

17. The microjet injector according to claim 11, wherein the elastic membrane and the nozzle block/the pressure chamber cylinder are engaged with each other such that the elastic membrane is disposed on an opposite side to one side of the nozzle block in which the microjet nozzle is formed, and a ring screw type nozzle holder is engagingly fitted around the pressure chamber cylinder so as to surround the elastic membrane and the nozzle block.

18. The microjet injector according to claim 17, further an elastic membrane holder interposed between the pressure chamber cylinder and the nozzle block so as to securely fix the elastic membrane, wherein the elastic membrane holder is constructed as a ring screw type member that has a pressing step formed on the inner peripheral surface thereof so as to press the periphery of the elastic membrane while surrounding the elastic membrane, so that the elastic membrane is inserted into the elastic membrane holder and then the elastic membrane holder is engagingly fitted around the bottom of the pressure chamber cylinder to cause the elastic membrane to be pressed against the pressure chamber cylinder.

19. The microjet injector according to claim 18 further comprising a ring-shaped rubber packing interposed between the elastic membrane and the pressure chamber cylinder.

20. The microjet injector according to claim 11, wherein the drug chamber formed in the nozzle holder has an inner accommodating space formed in a conical shape whose diameter is reduced as it goes toward the microjet nozzle.

21. The microjet injector according to claim 11, wherein the drug chamber comprises a drug supply passage formed therein so as to allow a drug solution to be charged into the drug chamber from the outside therethrough.

22. The microjet injector according to claim 11, wherein the transparent cap is made of a BK7 glass material.

23. The microjet injector according to claim 11, wherein the transparent cap have the shape of a convex lens which is bulged at the central portion thereof.

24. The microjet injector according to claim 11, wherein connecting adaptor further comprises an objective lens disposed therein so as to adjust the focal position of the laser beam.

25. The microjet injector according to claim 11, wherein the microjet nozzle is provided in two or more numbers.
